SEQUENCE LISTING (3 sequences)

SEQ ID NO: 1 - protein, 31 aa, Artificial Sequence
  amino acid sequence for the K34R GLP-1 (7-37) monomer
HAEGTFTSDVSSYLEGQAAKEFIAWLVRGRG

SEQ ID NO: 2 - DNA, 999 bp, Artificial Sequence
  nucleic acid sequence for the K34R GLP-1 (7-37) monomer codon optimized for E. coli
atgaaacgtcacgcggaaggcacctttacgtccgatgtgagctcttatctggaaggccaggcggccaaagaatttattgcctggctggtccgtggccgcggtaaacgtcatgccgaaggcacctttacgagcgacgtgagttcctacctggaaggtcaagcagctaaagaatttatcgcatggctggttcgtggccgcggcaaacgccacgctgaaggcacctttacgtctgatgtctcatcgtatctggaaggccaagccgcgaaagaatttatcgcctggctggtgcgtggccgcggcaaacgtcacgcagaaggcaccttcacgagtgacgttagctcttacctggaaggccaggccgccaaagaatttattgcttggttagttcgtggccgcggtaaacgccatgccgaaggcaccttcacgtccgatgtgagttcctatctggaaggccaagctgccaaagaatttatcgcttggttagtgcgtggccgcggaaagcgccacgcggaaggcaccttcacgtcagacgtctcatcgtacctggaaggccaggcggcgaaagaatttatcgcgtggttagtacgtggccgcggaaaacgccacgccgagggcacctttacgtcggatgttagctcttatctggaaggccaagcagcgaaagaatttattgcatggttggttcgtggccgcggaaagcgtcatgcagagggcacctttacgagcgatgtgagttcctacctggaagggcaggccgctaaggaatttatcgcgtggcttgttcgtggccgcggaaaacgtcatgcggagggcacctttacgtctgacgtctcatcgtatctggaaggccaggccgcgaaggaatttatcgcctggttagtccgtggccgcggcaagcgccatgcggagggcaccttcacgagcgacgttagctcttacctggaaggtcaagcggcgaaagaatttattgcgtggctggtccgtggtcgtggctaatga

SEQ ID NO: 3 - protein, 331 aa, Artificial Sequence
  amino acid sequence for K34R GLP-1 (7-37) concatemer
MKRHAEGTFTSDVSSYLEGQAAKEFIAWLVRGRGKRHAEGTFTSDVSSYLEGQAAKEFIAWLVRGRGKRHAEGTFTSDVSSYLEGQAAKEFIAWLVRGRGKRHAEGTFTSDVSSYLEGQAAKEFIAWLVRGRGKRHAEGTFTSDVSSYLEGQAAKEFIAWLVRGRGKRHAEGTFTSDVSSYLEGQAAKEFIAWLVRGRGKRHAEGTFTSDVSSYLEGQAAKEFIAWLVRGRGKRHAEGTFTSDVSSYLEGQAAKEFIAWLVRGRGKRHAEGTFTSDVSSYLEGQAAKEFIAWLVRGRGKRHAEGTFTSDVSSYLEGQAAKEFIAWLVRGRG

(19) United States
(12) Patent Application Publication
(10) Pub. No.: US 2020/0024321 A1
(43) Pub. Date: Jan. 23, 2020

(54) EXPRESSION AND LARGE-SCALE PRODUCTION OF PEPTIDES

(71) Applicant: Lupin Limited, Mumbai, Maharashtra (IN)

(72) Inventors: Sudharti GUPTA, Pune, Maharashtra (IN); Shardul Sumantrao SALUNKHE, Pune, Maharashtra (IN); Brajesh VARSHNEY, Pune, Maharashtra (IN); Rustom Sorab MODY, Pune, Maharashtra (IN)

(73) Assignee: Lupin Limited, Mumbai, Maharashtra (IN)

(21) Appl. No.: 16/496,026

(22) PCT Filed: Mar. 20, 2018
(86) PCT No.: PCT/IB2018/051842

§ 371 Date: Sep. 20, 2019

(30) Foreign Application Priority Data
Mar. 20, 2017 (IN) 201721009672

(51) Int. Cl. C07K 14/605; C12P 21/06

(52) U.S. Cl. CPC C07K 14/605; C12Y 304/17002; C12Y 304/21061; C12P 21/06

(57) ABSTRACT

The invention provides a method for the large-scale preparation of small peptides using recombinant DNA technology. Overexpression of small peptides, such as liraglutide precursor, as concatemers, improves the overall efficiency of the process due to increased yields per batch of the biologically active peptide. Digestion of these concatemers by combinations of specific enzymes yields the desired peptide monomer in large quantities. More particularly, the invention relates to the production of recombinant peptide precursor of liraglutide

### FIELD OF THE INVENTION

The present invention pertains to a process for the large scale preparation of a biologically active recombinant peptide in a suitable host by overexpressing it as a concatemer having specific intervening Kex2 protease and Carboxypeptidase B cleavage sites separating each monomer. Sequential digestion of the expressed multimer by Kex2 protease followed by carboxypeptidase yields the desired monomeric peptide in large quantities.

### BACKGROUND OF THE INVENTION

Glucagon-like peptide-1 (GLP-1), a product of the glucagon gene, is an important gut hormone known to be the most potent insulinotropic substance. It is effective in stimulating insulin secretion in non-insulin dependent diabetes mellitus (NIDDM) patients. Furthermore, it potently inhibits glucagon secretion and due to these combined actions it has demonstrated significant blood glucose lowering effects particularly in patients with NIDDM. A number of FDA approved GLP-1 analogs are available, for instance, exenatide (Byetta in 2005, Bydureon in 2012), albiglutide (Tanzeum in 2014), dulaglutide (Trulicity in 2014) and liraglutide (Victoza in 2010, Saxenda in 2014).

Liraglutide is an acylated derivative of the GLP-1 (7-37) that shares a 97% sequence homology to the naturally occurring human hormone by virtue of a substitution of lysine at position 34 by arginine (K 34R). It contains a palmitoylated glutamate spacer attached to e-amino group of Lys26. The molecular formula of liraglutide is C₁₇₂H₂₆₅N₄₃O₅₁ while its molecular weight is 3751.2 daltons.

Liraglutide was developed by Novo Nordisk (U.S. Pat. No. 6,268,343) as Victoza (FDA approval 2010) to improve glycemic control in adults with type 2 diabetes mellitus and as Saxenda (FDA approval 2014) for chronic weight management in obese adults in the presence of at least one weight-related comorbid condition. The peptide precursor of liraglutide was produced by recombinant expression in Saccharomyces cerevisiae.

Several chemical (solid-phase syntheses) and biological (recombinant) syntheses for the preparation of GLP-1 analogues have been described in the art.

Recombinant synthesis in simple hosts like E. coli or yeasts are plagued by either poor expression levels or high expression levels with scanty yields attributed to host degradative enzymes. This degradation has been overcome by the use of fusion tags or carriers like the histidine-tag, glutathione-S-transferase (GST), maltose binding protein, NusA, thioredoxin (TRX), small ubiquitin-like modifier(SUMO) and ubiquitin (Ub), which brings about safe delivery of the desired peptide. Expression of large fusion protein tags often leads to drop in overall yields and recovery of protein of interest, which is obtained after removal of the high molecular weight fusion partner from the peptides. Excision of the fusion tags by cleavage at specific sites either chemically (like CNBr) or by enzymatic methods confers inherent advantages pertaining to enhanced selectivity and specificity along with benign reaction conditions that lowers side reactions and helps to maximize yields.

U.S. Pat. No. 8,796,431 describes a process for producing a fusion peptide comprising an affinity tag, a cleavable tag and the peptide of interest (GLP-1 and liraglutide). Despite the ease and efficiency of purification via affinity chromatography, reduced overall yields were obtained.

The limitation of the fusion or carrier peptide approach has been overcome by expressing multiple repeats of the peptide of interest (POI) with intervening cleavage sites leading to respectable yields.

Thus, WO95/17510 discloses a method for producing GLP-1 (7-36) or its analogs using more than two consecutive DNA sequences coding for GLP-1 (7-36) which after expression was digested with enzymes like trypsin or clostripain and carboxypeptidase B or Y under suitable conditions to provide monomers. A similar strategy has been described in U.S. Pat. No. 7,829,307 for the preparation of GLP-2 peptides. U.S. Pat. No. 5,506,120 describes a process for preparing a concatemer of vasointestinal peptide (VIP) having alternate excisable basic dipeptide sites that was expressed in a mutant B. subtilis strain displaying less than 3% protease activity compared to the wild strain.

The present invention involves the preparation of the liraglutide peptide precursor K34R GLP-1 (7-37), the mGLP peptide, in a suitable host, such as E. coli, B. subtilis etc using its concatemer with intervening excision sites, thus reducing the total number of steps in obtaining the POI. Further, excision at the alternating di peptide cleavage sites simultaneously with kex2 protease and carboxypeptidase B allow preparation of the authentic peptide precursor without any extra terminal amino acid.

### SUMMARY OF THE INVENTION

In an embodiment, a concatemeric DNA construct for producing a peptide of SEQ ID 1, wherein the concatemeric DNA construct comprises:
a. DNA construct encoding a peptide of SEQ ID 1, codon optimized for expression in a suitable host;
b. wherein each unit of (a) is linked at its 3⁻ end to a monomeric or polymeric codon optimized spacer DNA sequence to encode for monomeric or polymeric units of the amino acids X₁—X₂,
   wherein X₁ is Lys or Arg and X₂ is Lys or Arg;
c. obtaining concatemeric DNA construct for cloning into a suitable host capable of being expressed as multimers of SEQ ID 1; and
d. obtaining multimers of SEQ ID 1, and treating with a combination of at least two proteases to obtain monomeric units of SEQ ID 1.

In an embodiment, expressing the concatemeric DNA construct to obtain multimers of peptide of SEQ ID 1 in the form of inclusion bodies.

In another embodiment, a process for producing a peptide of SEQ ID 1, the process comprising:
a. obtaining a codon optimized concatemeric DNA construct encoding for multimers of peptide of SEQ ID 1 for expression in a suitable host;
b. cloning concatemeric DNA construct of (a) into a suitable vector for expression in a suitable host;
c. expressing the concatemeric DNA construct of (a) to produce multimers of peptide of SE Q ID 1 as inclusion bodies;
d. simultaneously or sequentially contacting multimeric units of (c) with at least two proteases to obtain the peptide of SEQ ID 1.

In a further embodiment, cloning the concatemer in a prokaryotic or eukaryotic host using two or more inducers.

In a further embodiment, contacting the multimers of peptide of SEQ ID 1 simultaneously or sequentially with at least two proteases to obtain the peptide of SEQ ID 1.

In an embodiment, the present invention provides a process for producing the peptide precursor for liraglutide on a large scale by using its concatemer having alternate dipeptide Lys-Arg (K R) cleavage sites, excisable by sequential action of specific enzymes to release the biologically active monomer.

In another embodiment, a concatemeric gene containing 9-15 repeats of the gene for liraglutide precursor peptide having alternate K R sites was synthesized and then cloned into a suitable expression vector. Transformation of E. coli with the recombinant vector and its expression led to the peptide multimer as inclusion bodies.

In a further embodiment, the invention relates to a process for producing a biologically active GLP-1 (7-37), the process comprising:
a. obtaining a concatemeric gene construct containing 9-15 repeats of K34R GLP-1 (7-37) gene with each adjacent repeat separated by a cleavable K R site
b. cloning the above concatemeric construct into E. coli
c. expressing the concatemeric gene in E. coli
d. isolating the expressed protein from the cell culture in the form of inclusion bodies
e. solubilizing the inclusion bodies under optimum conditions
f. digesting the solubilized inclusion bodies under optimal conditions by sequentially subjecting them to specific enzymes essentially consisting of Kex2 protease (kexin) and carboxypeptidase B (CPB)

### BRIEF DESCRIPTION OF ACCOMPANYING FIGURES

FIG. 1 gives a schematic representation of the concatemer strategy with mGLP peptide as an example.

FIG. 2 shows the SDS PAGE gel picture of the E. coli concatemer clones displaying a high level expression of ˜35 kDa.

FIG. 3 illustrates the digestion profile of K34R GLP-1(7-37) inclusion bodies using varied concentrations of kex2 protease.

FIG. 4 illustrates the CPB digestion profile of kex2 protease-digested inclusion bodies

### DETAILED DESCRIPTION OF THE INVENTION

As used herein, the term ‘small peptide_or ‘peptides_refers to those having molecular weight ranging from about 2 to 10 kDa, used as a bio-therapeutic or for diagnostic and research purposes, wherein the preferred peptide is the peptide precursor for liraglutide, namely, K34R GLP-1 (7-37), the mGLP. The above-mentioned precursor contains amino acid residues from 7 to 37 of the glucagon-like peptide-1 (GLP-1) wherein the Lys at position 34 in the naturally occurring GLP-1 is substituted by Arg.

Especially in case of low molecular weight peptides, like the desired peptide, recombinant technology techniques are used to further enhance yield by expressing tandem gene repeats of the desired peptide that have been referred to herein as :concatemer’ which is defined as a long continuous DNA molecule that contains serially linked multiple copies of a smaller DNA sequence that codes for a monomer of the desired peptide. A concatemer may comprise 2-20 repeats of the monomer.

In the concatemer, individual DNA sequences coding for the monomer were separated by short cleavable dipeptidyl spacer sequences between every monomeric units. Many inactive precursors of bioactive peptides contain processing signal sequences made of a pair of basic dipeptides like Arg-Arg, Lys-Lys, Arg-Lys, Lys-Arg that are processed by specific enzymes to give the physiologically active peptides. Several proteases are known to show strict primary and secondary specificities to the above mentioned dipeptides and cleave precisely at the C- or N-terminus of or between the dipeptide. Particularly, this method is effective only when the desired peptide does not contain such a sequence recognizable by the excising enzyme. The preferred peptide K34R GLP-1 (7-37) being free of such basic di peptides in its sequence is an excellent candidate for the above method.

In the present invention, a concatemeric gene construct possessing intervening codons for the requisite excision sites was synthesized and inserted into a suitable expression vector. As used herein, the term ‘expression vector_refers to a DNA molecule used as a vehicle to artificially carry foreign genetic material into bacterial cell, where it can be replicated and over-expressed.

The concatemeric gene construct was placed downstream of a T7 promoter in the expression vector. As used herein, the term ‘promoter_refers to a regulatory region of DNA usually located upstream of the inserted gene of interest, providing a control point for regulated gene transcription.

For cloning, suitable host cells such as E. coli host cells were transformed by the recombinant expression vector. As used herein, an ‘E. coli host_refers to E. coli strains ranging from BL21, BL21 DE3, BL21 Al and others which are routinely used for expression of recombinant proteins.

In another embodiment, the expressed concatemer was ‘isolated from the cell culture_by one or more steps including lysing of the cells using a homogenizer or a cell press, centrifugation of the resulting homogenate to obtain the target protein as insoluble aggregates.

In an embodiment, the concatemer was expressed as insoluble inclusion bodies that inherently possessed specific dipeptide sites which, upon digestion with specific enzymes, released the desired monomeric peptide precursors. In a preferred embodiment, the intervening Lys-Arg (K R) sites were cleaved using sequential action of kex2 protease and carboxypeptidase B.

In another embodiment, the invention relates to a process of producing a biologically active GLP-1 (7-37), the process comprising:
a. creating a concatemeric gene construct containing 9 - 15 repeats of K34R GLP-1 (7-37) gene with each repeat separated from the adjacent one by codons for the K R dipeptide
b. cloning the above concatemeric construct into E. coli using a suitable expression vector
c. expressing the concatemeric gene in E. coli by inducing with arabinose and IPT G
d. isolating the expressed protein from the cell culture in the form of inclusion bodies
e. solubilizing the inclusion bodies at optimal conditions
f. digesting the solubilized inclusion bodies under optimal conditions by sequentially subjecting them to specific enzymes essentially consisting of kex2 protease (kexin) and carboxypeptidase B (CPB)

Experimental Section

K34R GLP-1 (7-37) was produced by recombinant DNA technology using genetically engineered E. coli cells. The E. coli cells were cultured and concatemers of the peptide precursor for liraglutide were obtained in the form of inclusion bodies, post induction. Inclusion bodies were processed by (subjected to) solubilization and sequential digestion to release the biologically active K34RGL P-1 (7-37) monomers.

### EXAMPLE 1

### Synthesis of Concatemer DNA

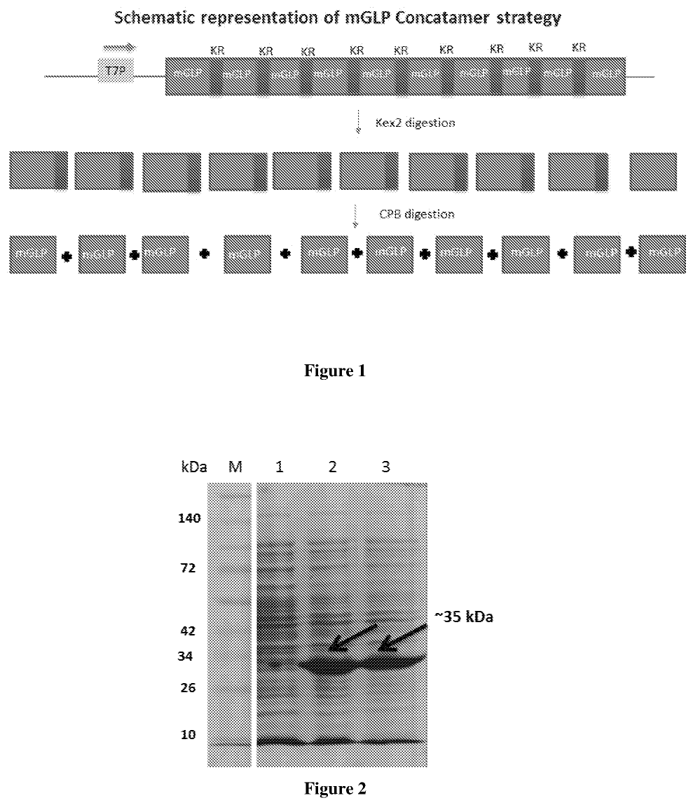

The nucleotide sequence derived from the amino acid sequence for K34R GLP-1 (7-37) monomer (SEQ ID 1) was codon optimized for E. coli (SEQ ID 2) to synthesize the K34R GLP-1 (7-37) concatemer (SEQ ID 3) as illustrated in FIG. 1.

### EXAMPLE 2

### Cloning of GLP Concatemer in pET 24a Expression Vector

The concatemer was synthesized and cloned into pET24a vector within the cloning sites, Nde I and Hind III. The vector pET24a possesses a strong T7 promoter for the expression of recombinant protein and a kanamycin resistance gene for selection and screening. The digested pET24a vector was ligated to the concatemer to provide the recombinant vector which was used to transform the E. coli host. The clones were screened by colony PCR and confirmed by restriction digestion with Nde I and Hind III and sequence analysis of the clone.

### EXAMPLE 3

### Expression of Concatemeric Protein

E. coli BL21 A1 cell line was used as the expression host. Other cell lines that may be used include BL21 DE3 or any other cell line that contains the T7 RNA polymerase. BL21 A1 cells transformed with the recombinant pET24a-GLP concatemer were induced (OD₆₀₀˜1) with 13 mM arabinose and 1 mM IPTG. The cells were harvested about 4 hours after induction. Determination of expression levels by SDS PAGE analysis of the whole cell lysate showed the presence of a ˜35 kDa band for the multimeric precursor peptide (FIG. 2, lanes 3, 4).

### EXAMPLE 4

### Solubilization of Inclusion Bodies

The cell lysate was further homogenized by sonication and centrifuged to separate inclusion bodies and soluble fractions. A bout 0.125 g inclusion bodies were weighed and dissolved in 3.0 mL of 2% SDS and 1.2 mL of 500 mM HEPES buffer (pH 7.5) diluted with milliQ water to make the volume to 6 mL. Complete solubilization (15-30 min) of the inclusion bodies was carried out by vortexing followed by centrifugation to obtain the K34R GLP-1 (7-37) multimer molecules in the supernatant. The solubilized inclusion bodies were further diluted 10 times in a final buffer composition of 50 mM HEPES, pH 7.5, 10 mM CaCl₂ and 2% Triton-X -100.

### EXAMPLE 5

### Protease Digestion with kex2 Protease and Carboxypeptidase B

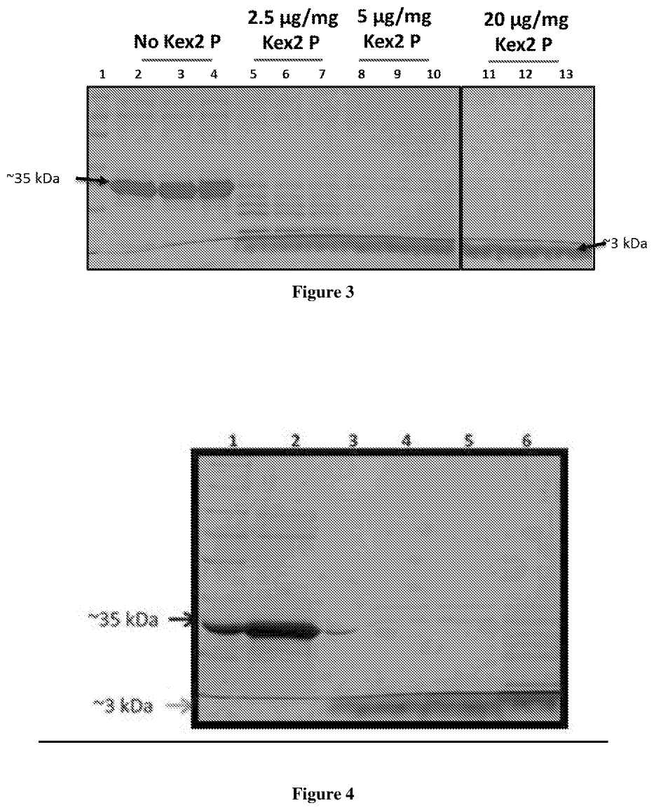

Protease digestion studies were carried out independently using 2.5, 5 and 20 ι g of kex2 protease (kex2 P) per mg of solubilized inclusion bodies for 20-28 h at room temperature. A band at 3 kDa observed by SDS PAGE (FIG. 3, lane 10 and 13) pertained to the monomer. Optimizing the quantities of kex2 protease by lowering from 20 ι g to 5 ι g and further to 2.5 ι g per mg of solubilized inclusion bodies showed complete digestion at 5 and 20 ι g and partial digestion at 2.5 ι g of kex2 protease used, with extended incubation with Kex2 protease for about 24-28 h. (FIG. 3).

A similar experiment was carried out with digestion of solubilized inclusion bodies with 5, 10 and 20 ι g of kex2 protease per mg of solubilized inclusion bodies for 16 h at room temperature. This was followed by further addition of 5 ι L (0.67 U/mL carboxypeptidase B (CPB) per mg of solubilized inclusion bodies at 37 éC for 2 hours. The resulting digestion mixture was analyzed by SDS PAGE (FIG. 4). The same was ascertained by comparison of its R P-H PLC peaks with that of a commercial GLP peptide from Sigma (data not shown).

### DETAILED DESCRIPTION OF FIGURES

FIG. 1: Schematic representation of concatemer strategy with GLP precursor peptide (mGLP peptide) as an example. The KR is a dipeptide which acts as recognition and cleavage site for kex2 protease enzyme. The kex2 enzyme will cleave the concatemer at the C terminus of the dipeptide resulting into peptide monomers along with the dipeptide, except last monomer. The dipeptides are removed through CPB digestion which specifically removes Lysine and Arginine residues at the C terminus.

FIG. 2: SDS PAGE analysis of whole cell lysate of E. coli concatemer clones. High level expression of multimeric mGLP is observed at ˜35 kDa level.

Lane 1: Molecular weight marker

Lane 2: Uninduced whole cell lysate of mGLP concatemer

Lane 3: Induced whole cell lysate of mGLP concatemer clone #1

Lane 4: Induced whole cell lysate of mGLP concatemer clone #2

FIG. 3: Optimization of kex2 protease digestion of mGLP inclusion bodies. As seen in FIG. 5 ι g and 20 ι g of Kex2 protease completely digested inclusion bodies to ˜3 kDA mGLP peptide, while 2.5 ι g of Kex2 protease partially digested the inclusion bodies, where a ladder of differentially digested peptide is visible.

Lane 1: Molecular weight marker

Lane 2 ⁻ mGLP (concatemer) undigested_20 h

Lane 3 ⁻ mGLP (concatemer) undigested_24 h

Lane 4 ⁻ mGLP (concatemer) undigested_28 h

Lane 5 ⁻ mGLP (concatemer) +2.5 ≈g of Kex2 protease/mg of mGLP concatemer⁻ 20 h

Lane 6 ⁻ mGLP (concatemer) +2.5 ≈g of Kex2 protease /mg of concatemer⁻ 24 h

Lane 7 ⁻ mGLP (concatemer) +2.5 ≈g of Kex2 protease/mg of mGLP concatemer⁻ 28 h

Lane 8 ⁻ mGLP (concatemer) +5 ≈g of Kex2 protease/mg of mGLP concatemer⁻ 20 h

Lane 9 ⁻ mGLP (concatemer) +5 ≈g of Kex2 protease/mg of mGLP concatemer⁻ 24 h

Lane 10 ⁻ mGLP (concatemer) +5 ≈g of Kex2 protease/mg of mGLP concatemer⁻ 28 h

Lane 11 ⁻ mGLP (concatemer) +20 ≈g of Kex2 protease/mg of mGLP concatemer⁻ 20 h

Lane 12 ⁻ mGLP (concatemer) +20 ≈g of Kex2 protease/mg of mGLP concatemer⁻ 24 h

Lane 13 ⁻ mGLP (concatemer) +20 ≈g of Kex2 protease/mg of mGLP concatemer⁻ 28 h

FIG. 4: Kex2 protease digestion of mGLP inclusion bodies followed by CPB treatment.

Lane 1: Molecular weight marker

Lane 2 ⁻ mGLP (concatemer) undigested - 16 h

Lane 3 ⁻ No loading

Lane 4 ⁻ mGLP (concatemer) +20 ≈g of Kex2 protease/mg of mGLP concatemer⁻ 16 h

Lane 5 ⁻ mGLP (concatemer) +10 ≈g of Kex2 protease/mg of mGLP concatemer⁻ 16 h

Lane 6 ⁻ mGLP (concatemer) +5 ≈g of Kex2 protease/mg of mGLP concatemer⁻ 16 h

Sequences

Sequence ID 1
HAEGTFTSDVSSYLEGQAAKEFIAWLVRGRG
Sequence ID 2
ATGAAACGTCACGCGGAAGGCACCTTTACGTCCGATGTGAGCTCTTATCT
GGAAGGCCAGGCGGCCAAAGAATTTATTGCCTGGCTGGTCCGTGGCCGCG
GTAAACGTCATGCCGAAGGCACCTTTACGAGCGACGTGAGTTCCTACCTG
GAAGGTCAAGCAGCTAAAGAATTTATCGCATGGCTGGTTCGTGGCCGCGG
CAAACGCCACGCTGAAGGCACCTTTACGTCTGATGTCTCATCGTATCTGG
AAGGCCAAGCCGCGAAAGAATTTATCGCCTGGCTGGTGCGTGGCCGCGGC
AAACGTCACGCAGAAGGCACCTTCACGAGTGACGTTAGCTCTTACCTGGA
AGGCCAGGCCGCCAAAGAATTTATTGCTTGGTTAGTTCGTGGCCGCGGTA
AACGCCATGCCGAAGGCACCTTCACGTCCGATGTGAGTTCCTATCTGGAA
GGCCAAGCTGCCAAAGAATTTATCGCTTGGTTAGTGCGTGGCCGCGGAAA
GCGCCACGCGGAAGGCACCTTCACGTCAGACGTCTCATCGTACCTGGAAG
GCCAGGCGGCGAAAGAATTTATCGCGTGGTTAGTACGTGGCCGCGGAAAA
CGCCACGCCGAGGGCACCTTTACGTCGGATGTTAGCTCTTATCTGGAAGG
CCAAGCAGCGAAAGAATTTATTGCATGGTTGGTTCGTGGCCGCGGAAAGC
GTCATGCAGAGGGCACCTTTACGAGCGATGTGAGTTCCTACCTGGAAGGG
CAGGCCGCTAAGGAATTTATCGCGTGGCTTGTTCGTGGCCGCGGAAAACG
TCATGCGGAGGGCACCTTTACGTCTGACGTCTCATCGTATCTGGAAGGCC
AGGCCGCGAAGGAATTTATCGCCTGGTTAGTCCGTGGCCGCGGCAAGCGC
CATGCGGAGGGCACCTTCACGAGCGACGTTAGCTCTTACCTGGAAGGTCA
AGCGGCGAAAGAATTTATTGCGTGGCTGGTCCGTGGTCGTGGCTAATGA
Sequence ID 3
MKRHAEGTFTSDVSSYLEGQAAKEFIAWLVRGRGKRHAEGTFTSDVSSYL
EGQAAKEFIAWLVRGRGKRHAEGTFTSDVSSYLEGQAAKEFIAWLVRGRG
KRHAEGTFTSDVSSYLEGQAAKEFIAWLVRGRGKRHAEGTFTSDVSSYLE
GQAAKEFIAWLVRGRGKRHAEGTFTSDVSSYLEGQAAKEFIAWLVRGRGK
RHAEGTFTSDVSSYLEGQAAKEFIAWLVRGRGKRHAEGTFTSDVSSYLEG
QAAKEFIAWLVRGRGKRHAEGTFTSDVSSYLEGQAAKEFIAWLVRGRGKR
HAEGTFTSDVSSYLEGQAAKEFTAWLVRGRG

1. A concatemeric DNA construct for producing a peptide of SEQ ID 1, wherein the concatemeric DNA construct comprises:
a. DNA construct encoding a peptide of SEQ ID 1, codon optimized for expression in a suitable host
b. wherein each unit of (a) is linked at its 3 ⁻ end to a monomeric or polymeric codon optimized spacer DNA sequence to encode for monomeric or polymeric units of the amino acids X₁—X₂,
wherein X₁ is Lys or Arg and X₂ is Lys or Arg;
c. obtaining concatemeric DNA construct for cloning into a suitable host capable of being expressed as multimers of SEQ ID 1; and
d. obtaining multimers of SEQ ID 1, and treating with a combination of at least two proteases to obtain monomeric units of SEQ ID 1.

2. The concatemeric DNA construct of claim 1, wherein the concatemer comprises of at least about 6 monomeric units.

3. The concatemeric DNA construct of claim 1, wherein the DNA construct is at least about 500 bps.

4. The concatemeric DNA construct of claim 1, wherein the DNA construct is expressed in a prokaryotic or eukaryotic host.

5. A multimeric peptide of SEQ ID 1, obtainable from the DNA construct of claim 1.

6. A monomeric peptide of SEQ ID 1, obtainable from the DNA construct of claim 1.

7. A process for producing a peptide of SEQ ID 1, the process comprising:
a. obtaining a codon optimized concatemeric DNA construct encoding for multimers of peptide of SEQ ID 1 for expression in a suitable host;
b. cloning concatemeric DNA construct of (a) into a suitable vector for expression in a suitable host;
c. expressing the concatemeric DNA construct of (a) to produce multimers of peptide of SEQ ID 1 as inclusion bodies;
d. simultaneously or sequentially contacting multimeric units of (c) with at least two proteases to obtain the peptide of SEQ ID 1.

8. The process as claimed in claim 7, wherein the vector is a pET vector.

9. The process as claimed in claim 7, wherein at least two inducers are used to induce expression of the concatemeric DNA construct.

10. The process as claimed i n claim 7, wherein the inducers are arabinose and IPTG.

11. The process of claim 1, wherein the proteases are Kex2 protease and Carboxypeptidase B.

12. The process as claimed i n claim 7, wherein the contact with kex2 protease and carboxypeptidase B is simultaneous.

13. The process as claimed i n claim 7, wherein the contact with kex2 protease and carboxypeptidase B is sequential.
